(19) United States
(12) Patent Application Publication
(10) Pub. No.: US 2026/0131074 A1
(43) Pub. Date: May 14, 2026

(54) INTEGRATED CGM AND INSULIN PUMP SYSTEM WITH INDUCTIVE POWER TRANSFER

(71) Applicant: Tandem Diabetes Care, Inc., San Diego, CA (US)

(72) Inventor: Reza Farshchi, San Diego, CA (US)

(21) Appl. No.: 19/379,273

(22) Filed: Nov. 4, 2025

Related U.S. Application Data
(60) Provisional application No. 63/719,440, filed on Nov. 12, 2024.

(51) Int. Cl. A61M 5/142

(52) U.S. Cl. CPC A61M 5/14248; A61M 2202/0007; A61M 2205/3303; A61M 2205/3561; A61M 2205/3592; A61M 2205/8206; A61M 2205/8243; A61M 2209/084; A61M 2209/088

(57) ABSTRACT

Ambulatory infusion pump systems can integrate a CGM sensor with an ambulatory infusion pump via a pump holder or tray that releasably holds the pump and is configured to be worn by a user. An inductive coupling between the pump and the CGM can both provide power from the pump to the CGM sensor and facilitate communications between the CGM sensor and the pump. By combining the CGM and insulin pump into one integrated system with inductive power transmission, a more aesthetically pleasing, convenient and reliable system is provided. Diabetes management is simplified by consolidating two essential devices into one.

### PRIORITY CLAIM

The present application claims the benefit of U.S. Provisional Patent Application No. 63/719,440 filed November 12, 2024, which is hereby incorporated herein by reference in its entirety.

### TECHNICAL FIELD

The present disclosure relates generally to ambulatory infusion pumps and, more particularly, to operation of ambulatory infusion pumps in conjunction with a continuous glucose monitor.

### BACKGROUND

There are a wide variety of medical treatments that include the administration of a therapeutic fluid in precise, known amounts at predetermined intervals. Devices and methods exist that are directed to the delivery of such fluids, which may be liquids or gases, are known in the art.

One category of such fluid delivery devices includes insulin injecting pumps developed for administering insulin to patients afflicted with Type 1 or Type 2 diabetes. Some insulin injecting pumps are configured as portable or ambulatory infusion devices that can provide continuous subcutaneous insulin injection and/or infusion therapy as an alternative to multiple daily injections of insulin via a syringe or an insulin pen. Such pumps can be worn or carried by the user and may use replaceable cartridges. In some embodiments, these pumps may also deliver medicaments other than, or in addition to, insulin, such as glucagon, pramlintide, and the like. Examples of such pumps and various features associated therewith include those disclosed in U.S. Patent Publication Nos. 2013/0324928 and 2013/0053816 and U.S. Patent Nos. 8,287,495; 8,573,027; 8,986,253; and 9,381,297, each of which is incorporated herein by reference in its entirety.

Ambulatory infusion pumps for delivering insulin or other medicaments can be used in conjunction with blood glucose monitoring systems, such as continuous glucose monitoring devices (CGMs). A CGM device may consist of a sensor placed under the patient’s skin and affixed to the patient via an adhesive patch, a transmitter, and a monitor. A CGM device samples the patient’s interstitial fluid periodically (e.g., once every 1-5 minutes) to estimate blood glucose levels over time. CGMs are advantageous because they provide more frequent insights into a user’s blood glucose levels yet do not require a finger stick each time a reading is taken.

Ambulatory infusion pumps may communicate with a dedicated CGM directly via a wired connection or indirectly via a wireless connection using wireless data communication protocols to communicate with a separate device (e.g., a dedicated remote device or a smartphone). One example of integration of ambulatory infusion pumps with CGM devices is described in U.S. Patent Publication No. 2014/0276419, which is hereby incorporated by reference herein. Ambulatory infusion pumps typically allow the user or caregiver to adjust the amount of insulin or other medicament delivered by a basal rate or a bolus, based on blood glucose data obtained by a CGM device, and in some cases include the capability to automatically adjust such medicament delivery. For example, based on CGM readings, some ambulatory infusion pumps may automatically adjust or prompt the user to adjust the level of medicament being administered or planned for administration or, in cases of abnormally low blood glucose readings, reducing or temporarily ceasing insulin administration.

While integrations of CGM devices with insulin pumps provides a number of advantages, the CGM sensor and pump are separately attached to the body resulting in a cumbersome and visually unappealing dual device configuration. Both the pump and the CGM also need to each have a battery, processor, communications element, etc. In addition, in order to establish wireless communications between the devices a pairing procedure must be undertaken. Patients are therefore burdened with managing two distinct systems, each with its own power source and attachment method, leading to increased complexity, higher costs and a less streamlined user experience.

### SUMMARY

Embodiments disclosed herein are directed to ambulatory infusion pump systems that integrate a CGM sensor with an ambulatory infusion pump via a pump holder or tray that releasably holds the pump and is configured to be worn by a user. An inductive coupling between the pump and the CGM can both provide power from the pump to the CGM sensor and facilitate communications between the CGM sensor and the pump. By combining the CGM and insulin pump into one integrated system with inductive power transmission, a more aesthetically pleasing, convenient and reliable system is provided. Diabetes management is simplified by consolidating two essential devices into one.

In embodiments, an ambulatory infusion pump system can include an ambulatory infusion pump including a housing, a reservoir configured to contain a medicament, a pumping mechanism configured to deliver the medicament from the reservoir to a user, a battery, and an inductive charging transmitter coil. The system can further include a glucose sensor configured to measure a signal indicative of glucose levels of a user and including an inductive charging receiving coil. A pump holder can include a tray configured to releasably retain the ambulatory infusion pump and including a sensor recess configured to receive the glucose sensor. The sensor recess can be positioned on the tray such that the inductive charging transmitter coil is aligned with and can provide power from the battery of the ambulatory infusion pump to the inductive charging receiving coil in the glucose sensor for operation of the glucose sensor when the glucose sensor is in the sensor recess of the tray and the ambulatory infusion pump is retained on the tray.

In embodiments, an ambulatory infusion pump system can include an ambulatory infusion pump including a housing, a reservoir configured to contain a medicament, a pumping mechanism configured to deliver the medicament from the reservoir to a user, and a battery. A pump holder can include a tray configured to releasably retain the ambulatory infusion pump, the pump holder including a sensor recess configured to receive a glucose sensor. The sensor recess can be positioned on the tray such that the battery of the ambulatory infusion pump provides power to the glucose sensor for operation of the glucose sensor when the glucose sensor is in the sensor recess of the tray and the ambulatory infusion pump is retained on the tray.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is an embodiment of an ambulatory infusion pump for use with embodiments of the disclosure.

FIG. 2 is a block diagram of the ambulatory infusion pump of FIG. 1.

FIGS. 3A-3B are an alternate embodiment of an ambulatory infusion pump for use with embodiments of the disclosure.

FIG. 4 depicts a schematic representation of an infusion pump system according to an embodiment of the disclosure.

FIG. 5 depicts an infusion pump system according to an embodiment of the disclosure.

FIG. 6 depicts the pump holder of the infusion pump system of FIG. 5.

FIGS. 7A-7B depict the CGM sensor of the infusion pump system of FIG. 5.

FIG. 8 depicts a portion of the infusion pump of the infusion pump system of FIG. 5.

FIG. 9 depicts an exploded view of the infusion pump system of FIG. 5.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

The following detailed description should be read with reference to the drawings in which similar elements in different drawings are numbered the same. The drawings, which are not necessarily to scale, depict illustrative embodiments and are not intended to limit the scope of the invention.

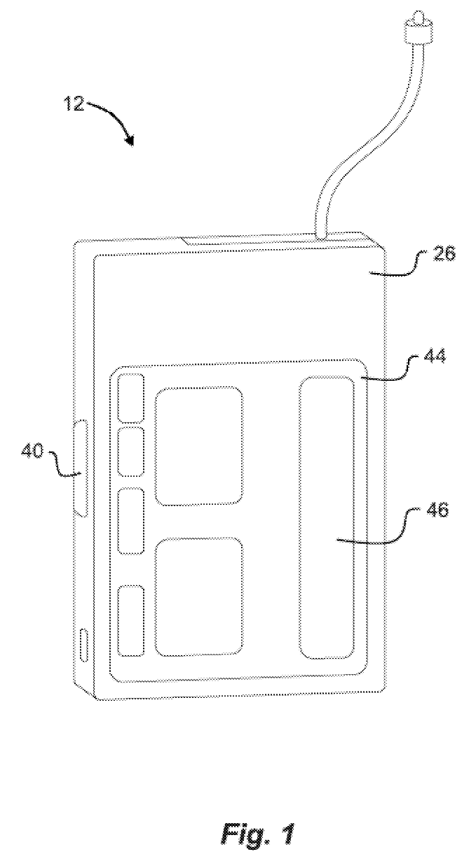

FIG. 1 depicts an exemplary medical device that can be used with embodiments of the disclosure. In this embodiment, the medical device is configured as a pump 12, such as an infusion pump, that can include a pumping or delivery mechanism and reservoir for delivering medicament to a patient and an output/display 44. The type of output/display 44 may vary as may be useful for a particular application. The output/display 44 may include an interactive and/or touch sensitive screen 46 having an input device such as, for example, a touch screen comprising a capacitive screen or a resistive screen. The pump 12 may additionally include a keyboard, microphone, or other input device known in the art for data entry, which may be separate from the display. The pump 12 may also include a capability to operatively couple to one or more blood glucose meters (BGMs) or continuous blood glucose monitors (CGMs) and/or one or more secondary devices such as a remote display, a remote control device, a laptop computer, personal computer, tablet computer, a mobile communication device such as a smartphone, a wearable electronic watch, smart ring, electronic health or fitness monitor, or personal digital assistant (PDA), a CGM display etc.

In one embodiment, the medical device can be a portable pump configured to deliver insulin to a patient. Further details regarding such pump devices can be found in U.S. Patent No. 8,287,495, which is incorporated herein by reference in its entirety. In other embodiments, the medical device can be an infusion pump configured to deliver one or more additional or other medicaments to a patient.

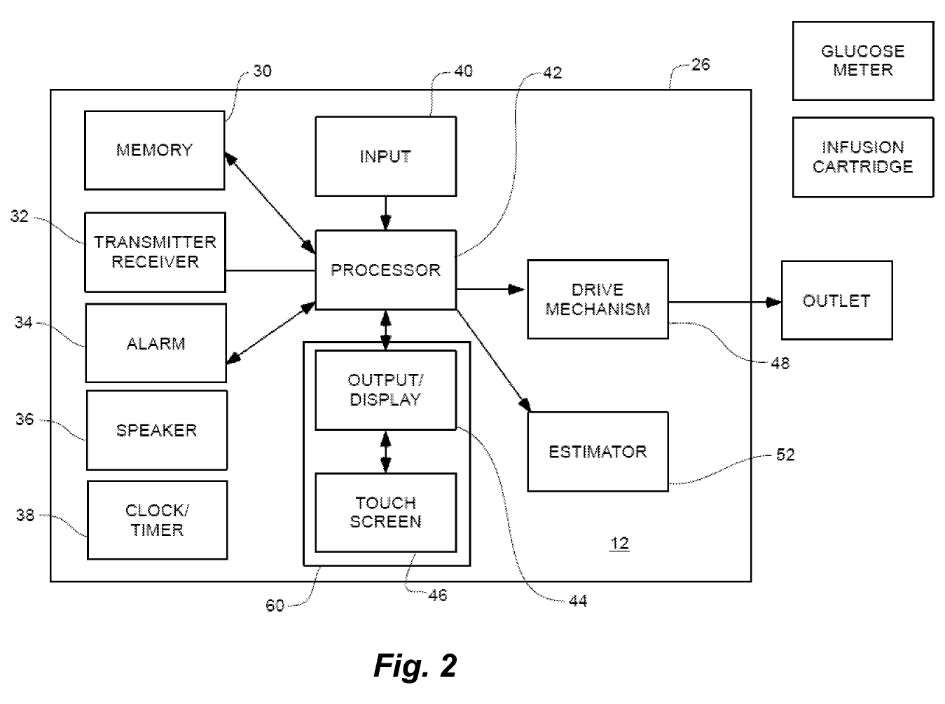

FIG. 2 illustrates a block diagram of some of the features that can be used with embodiments, including features that may be incorporated within the housing 26 of a medical device such as a pump 12. The pump 12 can include a processor 42 that controls the overall functions of the device. The infusion pump 12 may also include, e.g., a memory device 30, a transmitter/receiver 32, an alarm 34, a speaker 36, a clock/timer 38, an input device 40, a user interface suitable for accepting input and commands from a user such as a caregiver or patient, a drive mechanism 48, an estimator device 52, and a microphone (not pictured). One embodiment of a user interface as shown in FIG. 2 is a graphical user interface (GUI) 60 having a touch sensitive screen 46 with input capability. In some embodiments, the processor 42 may communicate with one or more other processors within the pump 12 and/or one or more processors of other devices, for example, a continuous glucose monitor (CGM), display device, smartphone, etc. through the transmitter/receiver. The processor 42 may also include programming that may allow the processor 42 to receive signals and/or other data from one or more input devices, such as sensors that may sense pressure, temperature and/or other parameters.

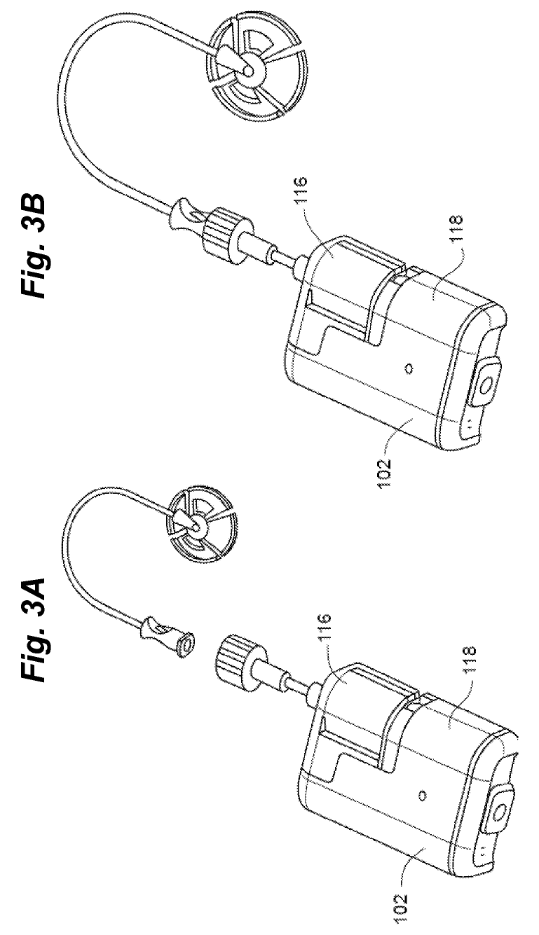

FIGS. 3A-3B depict a second infusion pump that can be used in conjunction with one or more embodiments of the ambulatory infusion pump system of the present disclosure. Pump 102 includes a pump drive unit 118 and a medicament cartridge 116. Pump 102 includes a processor 42 that may communicate with one or more processors within the pump 102 and/or one or more processors of other devices such as a remote device (e.g., a CGM device), a remote control device, or a consumer electronic device (e.g., laptop computer, personal computer, tablet computer, smartphone, electronic watch, smart ring, electronic health or fitness monitor, or personal digital assistant). The processor 42 may also include programming to receive signals and/or other data from an input device, such as, by way of example, a pressure sensor, a temperature sensor, or the like. Pump 102 also includes a processor that controls some or all of the operations of the pump. In some embodiments, pump 102 receives commands from a separate device for control of some or all of the operations of the pump. Such separate device can include, for example, a dedicated remote device or a consumer electronic device such as a smartphone having a processor executing an application configured to enable the device to transmit operating commands to the processor 42 of pump 102. In some embodiments, processor 42 can also transmit information to one or more separate devices, such as information pertaining to device parameters, alarms, reminders, pump status, etc. Such separate device can include any remote display, remote device, remote control, or a consumer electronic device as described previously.

Pump 102 can also incorporate any or all of the features described with respect to pump 12 in FIG. 2. In some embodiments, the communication is effectuated wirelessly, by way of example only, via a near field communication (NFC) radio frequency (RF) transmitter or a transmitter operating according to a “Wi-Fi” or Bluetooth® protocol, Bluetooth® low energy protocol or the like. Further details regarding such pumps can be found in U.S. Patent No. 10,279,106 and U.S. Patent Publication Nos. 2016/0339172 and 2017/0049957, each of which is hereby incorporated herein by reference in its entirety.

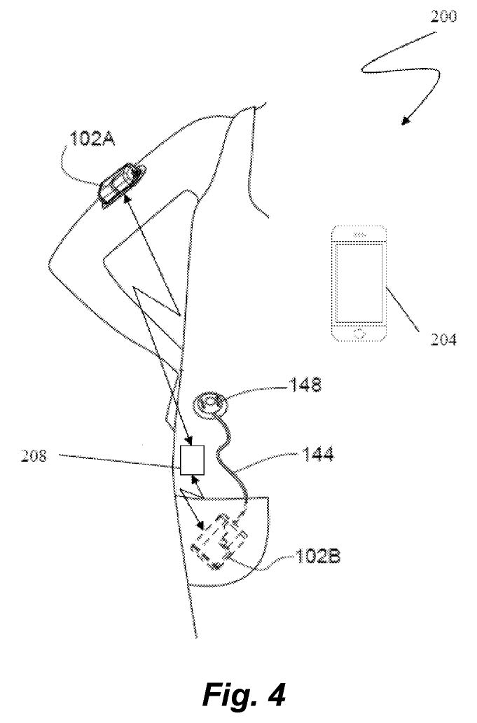

FIG. 4 depicts a schematic representation of a pump system 200 according to an embodiment of the disclosure. System 200 includes a user-wearable infusion pump such as pump 12 or pump 102 described above. In embodiments, a user can alternatively wear the pump 102A directly on the body or place the pump 102B in the user's pocket or other location near the body with infusion tubing 144 extending to an infusion set 148 on the user's body. The system 200 also includes a continuous glucose monitoring (CGM) sensor with a corresponding transmitter 208. The CGM sensor obtains measurements relating to glucose levels in the body and the transmitter can communicate that information to pump 102A/B. Pump 208 can then use the glucose data in making therapy determinations. The system can also include one or more devices such as a smartphone 204 or other multi-purpose consumer electronic device capable of operating a software application to communicate with and/or control the pump and, alternatively or additionally, a dedicated remote device designed specifically for use with pump 102A/102B. The smartphone 204 or other remote electronic device can in some embodiments also be capable of communication with CGM sensor/transmitter 208 and can calculate therapy commands for execution by the pump 102A/102B based on the glucose levels.

Although as described above infusion pumps and continuous glucose monitors are separate devices, there are disadvantages to this configuration. For example, the requirement for Bluetooth or other wireless communications between the pump and the CGM require the devices to be paired. This requires the user to undertake a complex pairing procedure to pair the two devices and errors can arise if the devices become unpaired or communication issues arise between the devices. The CGM sensor and the pump also require two different attachment sites and separate insertions into the user’s body. In addition, the separate devices each require their own batteries, processors, communications devices and associated circuitry. Embodiments described herein integrate the CGM with the ambulatory infusion pump to address each of these disadvantages.

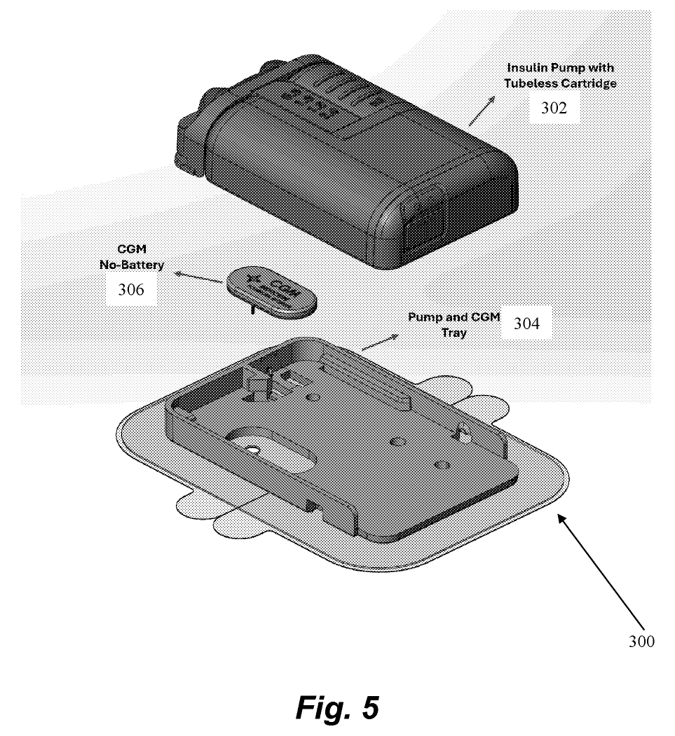

Referring to FIG. 5, an ambulatory infusion pump system 300 according to an embodiment of the disclosure is depicted. Ambulatory infusion pump system 300 is an integrated CGM and insulin pump system that can include a user-wearable infusion pump 302, a pump holder or tray 304 and a CGM sensor 306.

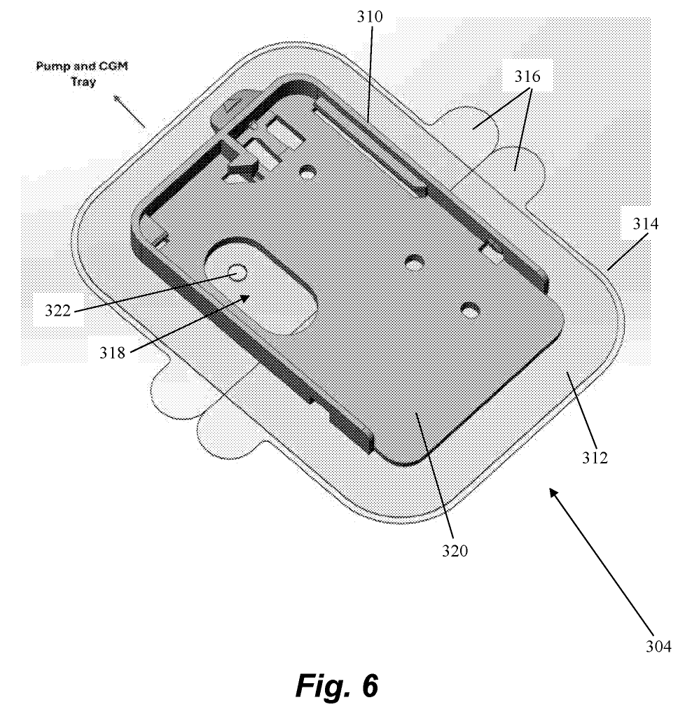

Pump holder 304 is also depicted in FIG. 6. Pump holder 304 can include a tray body 310 attached to an adhesive patch 312 that can releasably attach the pump holder 304 to a body of a user. A patch liner 314 comprising one or more pieces can include tabs 316 for removing patch liner 314 to expose the adhesive underside of adhesive patch 312 for attachment to the user. Tray body 310 can include a number of features related to guiding and releasably retaining an infusion pump on tray body 310, which are not described in detail herein. Further details regarding such features can be found in U.S Patent Publication Nos. 2024/0050650 and 2024/0226423, each of which is hereby incorporated herein by reference. Pump holder 304 can include a sensor recess 318 defined through a base surface 320 of tray body 310 sized and shaped to receive a CGM sensor such as sensor 306. In the depicted embodiment, the sensor recess 318 is configured such a top surface of the sensor 306 is flush with the base surface 320 of the tray body 310 to provide one continuous, flat surface on which the pump 302 will rest. The size and shape of sensor recess 318 can vary based on the configuration of a particular sensor. The location of sensor recess 318 on base surface 320 is selected to provide alignment of CGM sensor 306 with the pump 302 for optimal power and/or data transfer between CGM sensor 306 and pump 302. A wire aperture 322 can also be provided through adhesive patch 312 within sensor recess 318 to enable insertion of a sensor probe of the CGM sensor 306 through the pump holder 304 and into the user’s body.

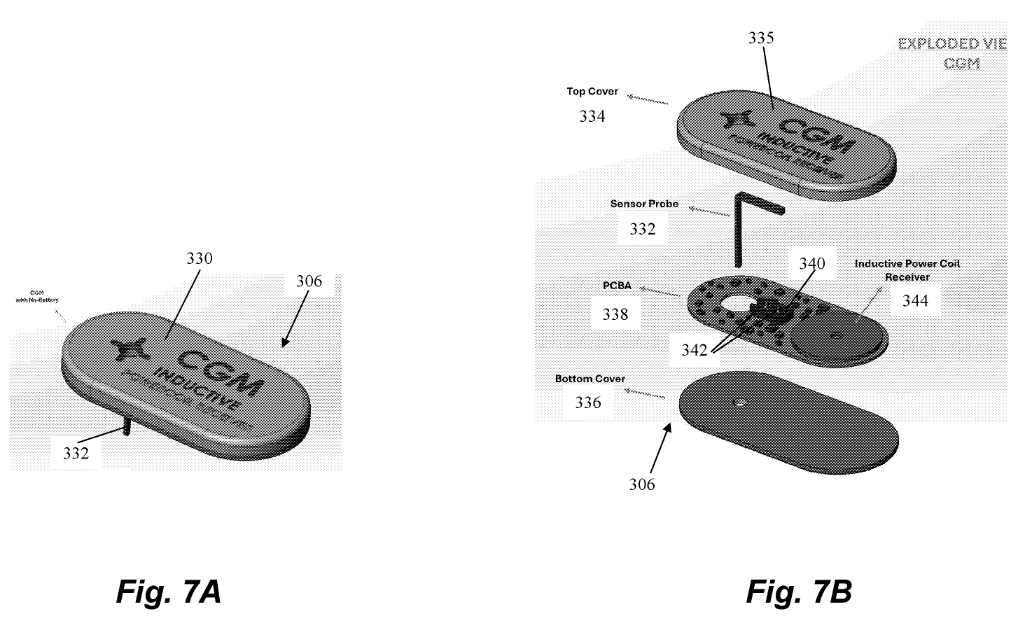

A CGM sensor 306 that can be used with a system 300 as disclosed herein is depicted in FIGS. 7A-7B herein. CGM sensor 306 can include sensor housing 330 with a sensor probe 332 extending from the housing that is configured to be inserted into the body of the user. Housing 300 may include a top cover 334, which includes the upper surface 335 that rests flush with base surface 320 of tray body 310 when the sensor 306 is inserted into the sensor recess 318, and a bottom cover 336 defining an opening therebetween in which a Printed Circuit Board Assembly (PCBA) 338 is housed. PCBA 338 can include a probe attachment 340 to which the probe 332 is attached and which can have one or more electrical contacts 342 that interface with the probe 332. PCBA 338 can also have an inductive power receiving coil 344 mounted thereon. This inductive power receiving coil 344 replaces the much larger battery traditionally used in similar CGM sensors, which significantly reduces the size and profile of the CGM sensor. This smaller form factor enables the pump holder 304 to more readily accommodate the sensor 306 to enhance patient comfort and discretion.

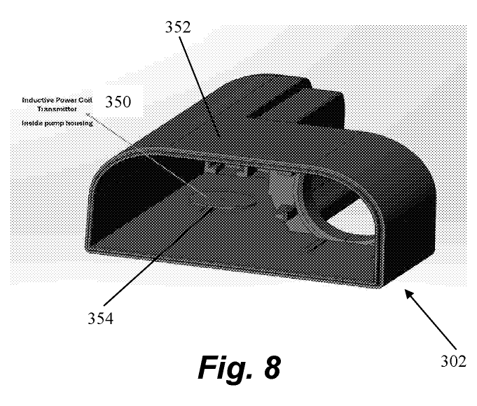

FIG. 8 depicts a portion of an ambulatory infusion pump 302 for use in an ambulatory infusion pump system 300 according to the disclosure. Ambulatory infusion pump 302 can include an inductive power transmitting coil 350 within a housing 352 of pump 302. In the depicted embodiment, inductive power transmitting coil 350 nests within a recess 354 defined in the housing 352. Additional circuitry would electrically couple the inductive power transmitting coil 350 with the electrical components of the pump 302, such as the battery and processor.

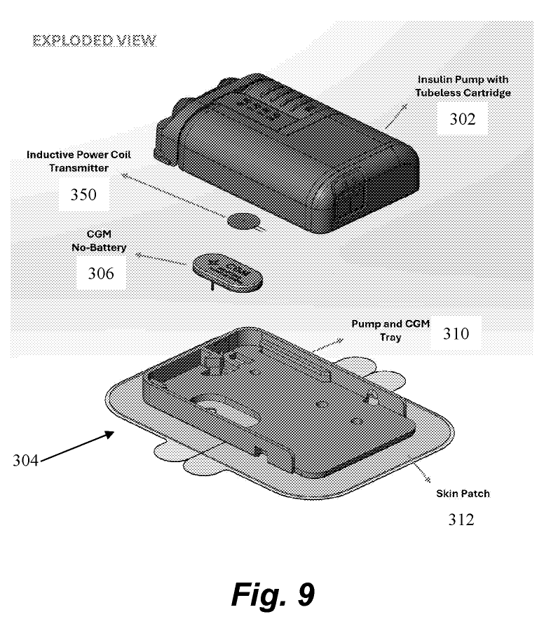

As can be seen in FIG. 9, inductive power transmitting coil 350 can be positioned within housing 352 such that the transmitting coil 350 is aligned with the inductive power receiving coil 344 within the CGM sensor 306 when the sensor is inserted into the sensor recess 318 in the tray body 310 and the pump 302 is attached to the pump holder 304.

In embodiments disclosed herein, a CGM sensor 306 is therefore powered by an inductive power coil receiver 344 that is energized by an inductive power coil transmitter 350 embedded in an insulin pump 302 that receives power from the internal battery in the pump 302. This eliminates the need for a traditional battery within the CGM sensor, reducing its size and cost. Elimination of the battery further eliminates the need for replacing the battery in the sensor, simplifying device maintenance and enhancing reliability since there will be no gaps in sensor readings due to a dead battery.

The insulin pump 302 and CGM sensor 306 are both housed within the tray 310 of the pump holder in a manner that ensures power transfer between the pump and the sensor while maintaining a sleek, unified appearance on the body. This reduces the number of visible devices on the body, providing a more discreet and visually appealing system.

In these embodiments, communications between the CGM sensor and pump can also be accomplished via the corresponding inductive coils. Pump can include internal circuitry to transmit the signals measured by the CGM sensor to the pump processor. The processor of pump can include programming sufficient to receive the CGM signals and to process those signals for use of the glucose levels indicated by the signals in providing therapy with the pump. In such embodiments, no Bluetooth or other wireless communications between the devices is required because the CGM signals are sent to the pump utilizing the inductive charging coil already present in the pump. As such, the CGM does not require a complex pairing procedure and wireless connectivity issues preventing transmission of CGM data will not occur. There are therefore further cost savings in the system because the CGM also does not need a processor, wireless communications elements and associated circuitry. The battery life of the pump will also be enhanced due to reduced power consumption from not having continual wireless communications with the CGM, because the power consumption of providing power to the CGM is significantly lower that the power required for wireless communications with the CGM.

In some embodiments, data obtained by the CGM sensor can be stored within a memory of the pump. This data could then be periodically synced to a smartphone application, the cloud or another external source to which the pump can periodically be connected. This can enhance data accessibility and historical data tracking, analysis and trends for patients and/or healthcare providers.

In embodiments, the pump will be able to determine if the connection with the CGM sensor has been disconnected because the pump will no longer be receiving data from the CGM and/or if the CGM loses power. In such circumstances, the pump can provide an alert informing the user that the CGM has become disconnected and/or has low or no power.

In some embodiments, the system can include a dual-purpose inserter (not pictured) to optionally allow for simultaneous insertion of both the CGM sensor and the insulin cannula for the pump into the body. This can simplify the insertion process for patients. Separate inserters can also be used. This may be beneficial in situations where the CGM sensor needs to be replaced more frequently than the cannula, or vice versa.

In embodiments, an ambulatory infusion pump system can include an ambulatory infusion pump including a housing, a reservoir configured to contain a medicament, a pumping mechanism configured to deliver the medicament from the reservoir to a user, a battery, and an inductive charging transmitter coil. The system can further include a glucose sensor configured to measure a signal indicative of glucose levels of a user and including an inductive charging receiving coil. A pump holder can include a tray configured to releasably retain the ambulatory infusion pump and including a sensor recess configured to receive the glucose sensor. The sensor recess can be positioned on the tray such that the inductive charging transmitter coil is aligned with and can provide power from the battery of the ambulatory infusion pump to the inductive charging receiving coil in the glucose sensor for operation of the glucose sensor when the glucose sensor is in the sensor recess of the tray and the ambulatory infusion pump is retained on the tray.

In some embodiments, the sensor recess is defined through a base surface of the tray.

In some embodiments, a top surface of the glucose sensor is flush with the base surface of the tray when the glucose sensor is disposed in the sensor recess.

In some embodiments, the sensor recess has a shape matching a perimeter shape of the glucose sensor.

In some embodiments, the pump holder further includes an adhesive patch configured to attach the tray to a body of a user, the adhesive patch including an aperture therethrough aligned with the sensor recess configured to enable insertion of a sensor probe of the glucose sensor through the aperture.

In some embodiments, the glucose sensor does not include an internal battery.

In some embodiments, the inductive charging transmitter coil is positioned adjacent a bottom surface of the ambulatory infusion pump that contacts the pump holder.

In some embodiments, the inductive charging transmitter coil is disposed in a recess adjacent the bottom surface of the ambulatory infusion pump.

In some embodiments, the ambulatory infusion pump and the glucose sensor can communicate without undergoing a wireless communication pairing procedure.

In some embodiments, communications between the ambulatory infusion pump and the glucose sensor are facilitated by the inductive charging transmitter coil and inductive charging receiving coil.

In embodiments, an ambulatory infusion pump system can include an ambulatory infusion pump including a housing, a reservoir configured to contain a medicament, a pumping mechanism configured to deliver the medicament from the reservoir to a user, and a battery. A pump holder can include a tray configured to releasably retain the ambulatory infusion pump, the pump holder including a sensor recess configured to receive a glucose sensor. The sensor recess can be positioned on the tray such that the battery of the ambulatory infusion pump provides power to the glucose sensor for operation of the glucose sensor when the glucose sensor is in the sensor recess of the tray and the ambulatory infusion pump is retained on the tray.

In some embodiments, the sensor recess is defined through a base surface of the tray.

In some embodiments, a top surface of the glucose sensor is flush with the base surface of the tray when the glucose sensor is disposed in the sensor recess.

In some embodiments, the sensor recess has a shape matching a perimeter shape of the glucose sensor.

In some embodiments, the pump holder further comprises an adhesive patch configured to attach the tray to a body of a user, the adhesive patch including an aperture therethrough aligned with the sensor recess configured to enable insertion of a sensor probe of the glucose sensor through the aperture.

In some embodiments, the glucose sensor does not include an internal battery.

In some embodiments, power from the battery of the ambulatory infusion pump is provided from an inductive charging transmitter coil in the ambulatory infusion pump to an inductive charging receiving coil in the glucose sensor.

In some embodiments, the inductive charging transmitter coil is positioned adjacent a bottom surface of the ambulatory infusion pump that contacts the pump holder.

In some embodiments, the inductive charging transmitter coil is disposed in a recess adjacent the bottom surface of the ambulatory infusion pump.

In some embodiments, the ambulatory infusion pump and the glucose sensor can communicate without undergoing a wireless communication pairing procedure.

Although embodiments described herein may be discussed in the context of the controlled delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to producenumerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Also incorporated herein by reference in their entirety are commonly owned U.S. Patent Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10/541,987; 10,569,016; 10,736,037; 10,888,655; 10,994,077; 11,116,901; 11,224,693; 11,291,763; 11,305,057; 11,458,246; 11,464,908; 11,654,236; 11,911,595; 12,138,425; 12,214,159; and 12,357,751 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0372995; 2021/0001044; 2021/0113766; 2022/0062553; 2022/0139522; 2022/0223250; 2022/0233772; 2022/0233773; 2022/0238201; 2022/0265927; 2023/0034408; 2022/0344017; 2022/0370708; ; 2022/0037465; 2023/0040677; 2023/0047034; 2023/0113545; 2023/0113755; 2023/0166033; 2023/0166037; 2023/0173170; 2023/0201452; 2023/0241314; 2023/0277765; 2023/0338653; 2023/0381406; 2024/0050650; 2024/0226423; 2024/0226424 and 2024/0277924; 2024/0399051; 2024/408303; 2024/0416032; 2024/0416033; 2025/0099674; 2025/0099675 2025/0099678; 2025/0099679; and 2025/0108162 and commonly owned U.S. Patent Applications Nos. 17/368,968; 17/896,492; 18/398,543; 18/962,169; 19/003,140; 19/003,164; 19/119,554; 19/134,333; 19/205,083; 19/220,426; 19/221,933; 19/225,150; and 19/252,256.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

1. An ambulatory infusion pump system, comprising:
an ambulatory infusion pump including a housing, a reservoir configured to contain a medicament, a pumping mechanism configured to deliver the medicament from the reservoir to a user, a battery, and an inductive charging transmitter coil; and
a pump holder including a tray configured to releasably retain the ambulatory infusion pump, the pump holder including a sensor recess configured to receive a glucose sensor including an inductive charging receiving coil,
wherein the sensor recess is positioned on the tray such that the inductive charging transmitter coil is aligned with and can provide power from the battery of the ambulatory infusion pump to the inductive charging receiving coil in the glucose sensor for operation of the glucose sensor when the glucose sensor is in the sensor recess of the tray and the ambulatory infusion pump is retained on the tray.

2. The ambulatory infusion pump system of claim 1, wherein the sensor recess is defined through a base surface of the tray.

3. The ambulatory infusion pump system of claim 2, wherein a top surface of the glucose sensor is flush with the base surface of the tray when the glucose sensor is disposed in the sensor recess.

4. The ambulatory infusion pump system of claim 1, wherein the sensor recess has a shape matching a perimeter shape of the glucose sensor.

5. The ambulatory infusion pump system of claim 1, wherein the pump holder further comprises an adhesive patch configured to attach the tray to a body of a user, the adhesive patch including an aperture therethrough aligned with the sensor recess configured to enable insertion of a sensor probe of the glucose sensor through the aperture.

6. The ambulatory infusion pump system of claim 1, wherein the glucose sensor does not include an internal battery.

7. The ambulatory infusion pump system of claim 1, wherein the inductive charging transmitter coil is positioned adjacent a bottom surface of the ambulatory infusion pump that contacts the pump holder.

8. The ambulatory infusion pump system of claim 7, wherein the inductive charging transmitter coil is disposed in a recess adjacent the bottom surface of the ambulatory infusion pump.

9. The ambulatory infusion pump system of claim 1, wherein the ambulatory infusion pump and the glucose sensor can communicate without undergoing a wireless communication pairing procedure.

10. The ambulatory infusion pump system of claim 1, wherein communications between the ambulatory infusion pump and the glucose sensor are facilitated by the inductive charging transmitter coil and inductive charging receiving coil.

11. An ambulatory infusion pump system, comprising:
an ambulatory infusion pump including a housing, a reservoir configured to contain a medicament, a pumping mechanism configured to deliver the medicament from the reservoir to a user, and a battery; and
a pump holder including a tray configured to releasably retain the ambulatory infusion pump, the pump holder including a sensor recess configured to receive a glucose sensor,
wherein the sensor recess is positioned on the tray such that the battery of the ambulatory infusion pump provides power to the glucose sensor for operation of the glucose sensor when the glucose sensor is in the sensor recess of the tray and the ambulatory infusion pump is retained on the tray.

12. The ambulatory infusion pump system of claim 11, wherein the sensor recess is defined through a base surface of the tray.

13. The ambulatory infusion pump system of claim 12, wherein a top surface of the glucose sensor is flush with the base surface of the tray when the glucose sensor is disposed in the sensor recess.

14. The ambulatory infusion pump system of claim 11, wherein the sensor recess has a shape matching a perimeter shape of the glucose sensor.

15. The ambulatory infusion pump system of claim 11, wherein the pump holder further comprises an adhesive patch configured to attach the tray to a body of a user, the adhesive patch including an aperture therethrough aligned with the sensor recess configured to enable insertion of a sensor probe of the glucose sensor through the aperture.

16. The ambulatory infusion pump system of claim 11, wherein the glucose sensor does not include an internal battery.

17. The ambulatory infusion pump system of claim 11, wherein power from the battery of the ambulatory infusion pump is provided from an inductive charging transmitter coil in the ambulatory infusion pump to an inductive charging receiving coil in the glucose sensor.

18. The ambulatory infusion pump system of claim 17, wherein the inductive charging transmitter coil is positioned adjacent a bottom surface of the ambulatory infusion pump that contacts the pump holder.

19. The ambulatory infusion pump system of claim 18, wherein the inductive charging transmitter coil is disposed in a recess adjacent the bottom surface of the ambulatory infusion pump.

20. The ambulatory infusion pump system of claim 11, wherein the ambulatory infusion pump and the glucose sensor can communicate without undergoing a wireless communication pairing procedure.
